(19) United States
(12) Patent Application Publication
(10) Pub. No.: US 2007/0183998 A1
(43) Pub. Date: Aug. 9, 2007

(54) Cosmetic composition

(75) Inventors: Takahiro Suzuki, Tokyo (JP); Toshiyuki Kani, Tokyo (JP); Mariko Okamoto, Tokyo (JP)

Correspondence Address: OLIFF & BERRIDGE, PLC, P.O. BOX 19928, ALEXANDRIA, VA, 22320, US

(73) Assignee: L'OREAL, PARIS (FR)

(21) Appl. No.: 11/636,527

(22) Filed: Dec. 11, 2006

Related U.S. Application Data
(60) Provisional application No. 60/754,196, filed on Dec. 28, 2005.

(51) Int. Cl. A61K 8/49; A61K 8/35

(52) U.S. Cl. 424/63; 424/64

(57) ABSTRACT

The present invention relates a cosmetic composition comprising: at least one pigment comprising: an elongated core, a red organic colorant coating at least partially the core, and at least one binder between the core and the organic colorant.

This non provisional application claims the benefit of U.S. Provisional Application No. 60/754,196 filed on Dec. 28, 2005.

### FIELD OF THE INVENTION

The present invention relates to a cosmetic composition and to a method for manufacturing a cosmetic composition.

### BACKGROUND

Some makeup cosmetic products, especially foundations, aim to cover up blemishes such as spots and freckles to create, over the naked skin, an ideal skin color which is different from the naked skin.

Therefore, some foundations contain ingredients with high covering ability, for example coloring pigments such as titanium or iron oxide, in order to improve the ability to cover up imperfections.

However, while the application of such foundations can cover up blemishes on the skin such as dullness, they can result in an unnaturally and powdery appearance overall.

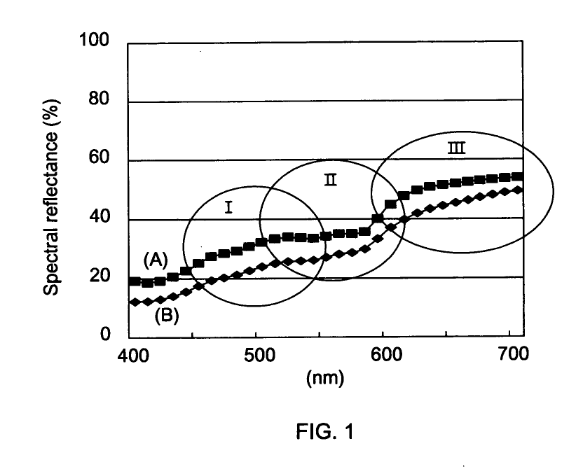

Human skin, in its original and unblemished state, has a natural healthy skin color with a sense of translucence which, when measured by spectral reflectance, is known to exhibit a curve as shown in FIG. 1(A) having a broad peak in a wavelength region (I) of 450-550 nm, a trough in a wavelength region (II) of 500-620 nm, and gradually increasing in a wavelength region (III) of 600-760 nm. The curve of FIG. 1 (A) is said to reflect a “natural and beautiful skin”.

However, skin which has been dulled by various types of damage including UV rays and the like is known to exhibit an overall decrease in the spectral reflectance, as well as a tendency for the features in above-mentioned wavelength regions to flatten out, as shown in FIG. 1 (B).

JP-A-H10-17437 discloses a foundation for obtaining natural and healthy-looking skin, by taking into account the changes in the trough in the spectral reflectance curve of region (II), and to restore this trough in the curve by including red colorants and yellow colorants in a specific proportion.

JP-A-2005-36010 also discloses a foundation for obtaining the trough in the spectral reflectance curve of region (II).

In JP-A-2005-36010, red or yellow colorants are used to restore the trough in region (II), while chromium oxide is used for providing peak in the wavelength region (I).

JP-A-2002-241212 discloses a foundation containing an interference pigment capable of obtaining natural finish with translucence.

JP-A-2004-315426 discloses a powder obtained by covering the surface of a titanium oxide-covered mica with a red organic colorant. The TiO₂ coated mica enhances the bright reflection on face, while the red organic colorant contributes to restore the spectral reflectance described above.

Although the composition disclosed in JP-A-2004-315426 may improve the appearance of skin, there was a problem to be solved for coating the TiO₂-mica.

The TiO₂-mica is a mica fragment having surface coated with titanium oxide. This titanium oxide-coated mica preferably has an average grain size of about 15-30 μm. The thickness of the mica is 200-300 nm and it has a high aspect ratio of about 50-100. On the other hand, the thickness of titanium oxide layer on the mica surface is such thick of about 150-450 nm on each side of mica surface. The thickened TiO₂-mica reduces the smoothness of foundation to spread on skin and gives creaky touch to the foundation.

Another potential problem of red colorant-TiO₂-mica composite is that the beautiful appearance of skin on face does not last for a long time. Adhesion of thick powder against skin is weak as reported in JP-A-2003-81767. The thick powder is hard to fit to skin and tends to be removed by sweat and sebum during wearing.

Furthermore, the binding of a colorant to TiO₂-mica is based on surface potentials of the colorant and powder. The surface potential of TiO₂ is charged positively, while ionized carboxyl residue of the colorant binds to the positive site of the surface of TiO₂. Thereby, the colorant can easily be removed from the surface of TiO₂ by attacking with acidic components such as fatty acids dissolved in sebum and sweat.

In JP-A-2004-315426, the inventors are describing that various surface treatments with polymers are available to coat on a red organic colorant-TiO₂-mica composite. However, those surface treatments are not available to stabilize the binding of a colorant with TiO₂.

Some amounts of red colorant at the surface of composite powder can be removed if wetted by sebum and sweat. The composite powder of JP-A-2004-315426 is not available in long wearing makeup products. That is, the colorant may be removed from the surface of TiO₂ during wearing or if it is comprised in liquid makeup products. Consequently, there is no available method for long wearing and for liquid makeup products to restore the spectral reflectance curve of the skin.

EP 1 586 295 discloses a mixture of composite pigments having different colors, with several organic colorants.

### SUMMARY

There is a need to benefit from a cosmetic product available for long wearing and for liquid makeup products to improve the appearance of skin and obtain a spectral reflectance curve close to the spectral reflectance curve of natural healthy skin.

The present invention may obviate this need.

Some exemplary embodiments of the invention are directed to a cosmetic composition comprising:
at least one pigment comprising:
   an elongated core, and
   a red organic colorant coating at least partially the core,
   at least one binder between the core and the organic colorant.

The term “elongated” means that the aspect ratio is greater than 1.

The pigment comprising a thin core and a red organic colorant may have a red organic colorant may have a red color a* in the CIE space L*a*b* comprised between 5 and 45.

In the CIE L*a*b* color space, the color value a* of the pigment is measured as follows. A certain amount of the pigment is filled in a glass cell with transparent flat bottom. The color values of a* of the pigment in the cell may be measured by use of a CR-200 (Minolta).

The at least one pigment of the invention may improve appearance of skin.

The elongated core may have an aspect ratio not less than 20, or even not less than 30. The higher aspect ratio of the core is effective on adhesion of pigment on skin and, therefore, effective on long wearing. Beside this, the higher aspect ratio is effective for increasing the reflection ratio of light from the skin, which is essential for restoring the personal beautiful skin from the dull color of damaged skin.

The binder may derive from one compound which molecule(s) includes at least two active sites to bind respectively with the colorant and the core. The binder may be based on at least a silicone compound, for example a methicone, and may have perfluoro residues, for example heptadecafluorodecyltriethoxysilane.

The organic colorant may comprise synthetic organic colorants and natural colorants, such as carmine. The organic colorant may be an anthraquinone-type colorant. The content of the red organic colorant may be 0.1%-5% by weight, relative to the total weight of the composition.

The core may be an inorganic core or an organic core.

There may be at least one intermediate layer between the organic colorant and the core.

The intermediate layer may include for example at least one metal oxide, chosen from titanium dioxide, zinc oxide, zirconium oxide, cerium oxide, iron oxide, aluminium oxide, chrome oxide, calcium oxide, silica, and magnesium oxide. The core may have a mean size of about 1-40 μm and more preferably about 5-20 μm.

The term “mean size” means the dimension given by the statistical grain size distribution curve at 50% population, termed D50. The mean size may be a number average determined by image analysis (electron microscopy).

The composition may comprise a fatty phase chosen among waxes, oils, gums, and/or paste-type fatty substances of plant, animal, mineral, synthetic or silicone origin and mixtures thereof.

The composition may be an emulsion, the proportion of the fatty phase in the composition may lie in the range 5% to 80% by weight, and for example 5% to 50% by weight, relative to the total weight of the composition.

In some exemplary embodiments, the core comprises sericite, the red organic colorant comprises carmine and the binder is based on a silicone. The pigment may be in the form of flakes having a carmine/methicone binder-sericite.

The weight ratio of carmine may range from 0.1% to 5%, the weight ratio of binder may range from 0.3% to 10% and the weight ratio of sericite may be range from 85% to 99.6%.

The percentage by weight, relative to the total weight of the composition, of sericite-carmine-binder may be range from 0.5% to 90%.

The cosmetic composition may be a blush, a base makeup, a foundation, for example a liquid foundation, powder foundation or a solid foundation.

The composition may be other makeup cosmetic composition such as a lip rouge, eyeshadow, eyebrow color or mascara.

The spectral reflectance curve of a skin made-up with the composition of the invention may have a broad peak in a wavelength region of 450-550 nm, a trough in a lower wavelength region of 500-620 nm, and gradually increasing in the wavelength region of 600-700 nm.

The term “trough” means that there is a reduction of the reflectance, for example at least 0.1%.

The present invention also relates to a method of making a cosmetic composition, wherein an organic colorant, for example a red organic colorant, is premixed and connected with a binder in a first time, and in a second time, the core is added to the organic colorant-binder.

A composition obtained by such a method is highly effective on long lasting. Moreover, such a composition has good repellent property and the conjugation between the core and the colorant is stable. For example, contact angles of water on the colorant-core with a binder may be higher than 100°.

Another object of the present invention is a method of making a cosmetic composition, wherein the surface of an elongated core is treated by a premixed organic colorant, for example a red organic colorant connected to a binder.

In these methods, the organic colorant may be carmine, the binder may be a silicone and the core may be sericite, for example.

The present invention also relates to a cosmetic method for restoring a natural and healthy beautiful skin, comprising application to the skin of a cosmetic composition such as defined above.

The invention also provides the use of a composition having soft focus effects in accordance with the invention for making up the skin, lips or the nails.

The present invention also relates to a cosmetic method for making up the damaged skin which has been dulled (the skin having a flat spectral reflectance curve without peaks) to the “natural and beautiful skin”, comprising application to the damaged skin of a cosmetic composition containing the at least one pigment according to the present invention.

This cosmetic method includes not only embodiments in which the cosmetic composition according to the present invention is applied directly to the skin, but also embodiments in which the composition of the present invention is applied over a coat.

The present invention thus also relates to a kit comprising:
a first cosmetic composition such as defined above, and
a second cosmetic composition, the two compositions being separately packaged and configured to be applied one onto the other.

Spectral Reflectance Curves of Skin

In the present invention, “natural and beautiful skin” refers to unblemished skin which has not been damaged by UV rays or the like, i.e. skin of a color having the spectral reflectance shown in FIG. 1(A) without strong highlighting.

As can be seen on FIG. 1(A), the spectral reflectance curve of “natural and beautiful skin” has a broad peak in a wavelength region (region I) of 450-550 nm, a trough in a lower wavelength region (region II) of 500-620 nm, and gradually increasing in the wavelength region (region III) of 600-700 nm. In contrast, skin which has been damaged and has a dull color, has a flattened spectral reflectance curve as shown on FIG. 1(B), which is less bumpy than (A) and has a lower overall reflectance.

Consequently, in the present invention, “restoring a natural and beautiful skin” refers to changing skin which has been damaged and has a flat spectral reflectance curve like that shown in FIG. 1(B) or similar skin such as to give it the above-described “natural and beautiful skin”.

Pigment

The at least one pigment comprised in the cosmetic composition according to the present invention is formed by coating a core with an organic colorant with aid of a binder.

While the content of the pigments, made in accordance with the invention, in the composition is not particularly restricted, it may be about 1-99% by weight, more preferably about 2-70% by weight, even more preferably about 3-30% by weight with respect to the entire weight of the composition.

Organic Colorant

The organic colorant contained in the at least one pigment of the present invention includes synthetic organic colorants and natural colorants generally classified as red. Synthetic organic colorants include, for example, dyes including azo-type dyes such as Red No. 505 and xanthan-type dyes such as Red No. 230 and Red No. 233, lakes such as Red No. 202, and organic pigments such as Red No. 228, or mixtures thereof. Natural colorants include, for example, carotenoide colorants such as Carthamin, flavonoid colorants including anthocyanidins and chalcones, quinine colorants including anthraquinones and naphthoquinones, and betacyanidin colorants such as betanin, or mixtures thereof.

Of these red organic colorants, it is preferable to use natural colorants, especially anthraquinone colorants, among which carminic acid (cochineal) or its aluminium lakes (carmine) are preferred.

Core

The core may be inorganic or organic. Examples of cores include talc, mica, synthetic mica, sericite, silica, titanium oxide, kaolin, Nylon® (Orgasol® by Atochem), powders of poly-β-alanine and polyethylene powders, Teflon, lauroyllysine, starches, boron nitride, tetrafluoroethylene polymer, hollow microspheres such as Expancel® (Nobel Industries), Polutrap® (Dow-coming) and silicone resin microbeads (such as Tospearls® by Toshiba), calcium carbonate, magnesium carbonate and magnesium hydroxide, hydroxyapatite, hollow silica microspheres (silica beads by Maprecos), glass or ceramic microcapsules, and metal soaps derived from organic carboxylic acids having 8-22 carbon atoms, preferably 12-18 carbon atoms, such as zinc, magnesium or lithium stearate, zinc laurate or magnesium myristate, or mixtures thereof.

Of these cores, it is preferable to use elongated cores with an aspect ratio not less than 20, especially synthetic mica (Sun Chemical) is preferred. While this core preferably has a mean size of about 1-40 μm and more preferably about 5-20 μm, those with a mean size of less than 1 μm are also acceptable.

The core may be a composite core, being covered at least partially by at least one layer of a metal oxide for example, titanium dioxide, zinc oxide, zirconium oxide, cerium oxide, iron oxide, aluminium oxide, chrome oxide, calcium oxide, silica, and magnesium oxide.

Binder

The binder may derive from a compound which molecules have at least two active sites to bind with colorant and core.

More particularly, the binder exhibits hydrophobic properties after combining with colorant(s) and filler(s). More particularly, it is cross-linked.

The binder may be based on at least a compound chosen from a non-limiting list comprising silicone compounds, for example silicone polymers, polymeric or oligomeric compounds or the like, and in particular from organosilanes, fluoroalkyl organosilanes and polysiloxanes, and also various couplers, such as couplers based on silanes, like for example alkoxysilane and chlorosilane, on titanates, on aluminates or on zirconates, and mixtures thereof.

In particular, the silicone compound may be chosen from:
the organosilanes (1) obtained from alkoxysilanes,
the modified or unmodified polysiloxanes (2) chosen from a non-limiting list comprising:
   the modified polysiloxanes (2A) comprising at least one radical chosen especially from polyethers, polyesters and epoxy compounds (these will be referred to as “modified polysiloxanes”),
   the polysiloxanes (2B) bearing, on a silicon atom located at the end of the polymer, at least one group chosen from a non-limiting list comprising carboxylic acids, alcohols and hydroxyl groups, and
   the fluoroalkyl organosilane compounds (3) obtained from fluoroalkylsilanes.

The organosilane compounds (1) may be obtained from alkoxysilane compounds represented by formula (I):
R¹ₐSi X₄₋ₐ (I)
in which:
R¹ represents C₆H₅—, (CH₃)₂ CH CH₂— or n- C_{b H}_{2b+1}- (in which b ranges from 1 to 18),
X represents CH₃O— or C₂H₅O—, and
a ranges from 0 to 3.

Specific examples of alkoxysilane compounds may include alkoxysilanes chosen from: methyltriethoxysilane, dimethyldiethoxysilane, phenyltriethyoxysilane, diphenyldiethoxysilane, methyltrimethoxysilane, dimethyldimethoxysilane, phenyltrimethoxysilane, diphenyldimethoxysilane, isobutyltrimethoxysilane, decyltrimethoxysilane and the like, in particular from methyltriethoxysilane, phenyltriethyoxysilane, methyltrimethoxysilane, dimethyldimethoxysilane and isobutyltrimethoxysilane, and even better still methyltriethoxysilane, methyltrimethoxysilane or phenyltriethyoxysilane.

The polysiloxanes (2) may especially correspond to formula (II): 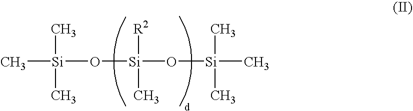
in which R² represents H— or CH₃— and d ranges from 2 to 450.

Among these polysiloxanes, those for which R² represents H are preferred.

The modified polysiloxanes (2A) may especially correspond to the following formulae:
(a¹) modified polysiloxanes bearing polyethers, represented by formula (III) 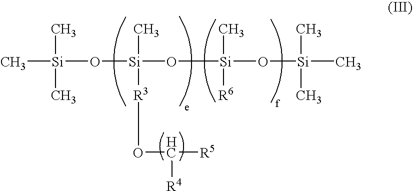
   in which R³ represents —(CH₂)h—; R⁴ represents —(CH₂)ᵢ—CH₃; R⁵ represents —OH, —COOH, —CH═CH₂, —C(CH₃)═CH₂ or —CH₂)ⱼ—CH₃; R represents —CH2)ₖ—CH₃; g and h ranging independently from 1 to 15; j and k ranging independently from 0 to 15; and e ranging from 1 to 50 and f ranging from 1 to 300,
(a²) modified polysiloxanes bearing polyesters, represented by formula (IV): 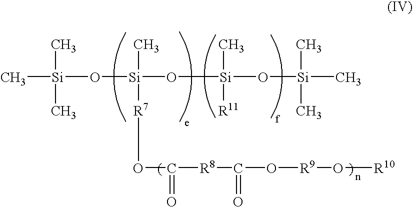
   in which R⁷, R⁸ and R⁹ independently represent —(CH2)_{q}—; R¹⁰ represents —OH; —COOH, —CH═CH₂, —C(CH₃)═CH₂ or —(CH₂)r—CH₃; R¹¹ represents —(CH₂)s—CH₃; n and q ranging independently from 1 to 15, r and s ranging independently from 0 to 15; e ranging from 1 to 50 and f ranging from 1 to 300,
(a³) modified polysiloxanes bearing epoxy radicals represented by formula (V): 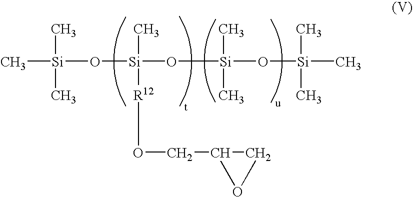
   in which R¹² represents —(CH₂)ᵥ—; v ranging from 1 to 15; t ranging from 1 to 50 and u ranging from 1 to 300; or mixtures thereof.
   Among the modified polysiloxanes (2A), the modified polysiloxanes bearing polyethers of formula (III) are preferred.
   The polysiloxanes modified on the end portion (2B) may correspond to formula (VI): 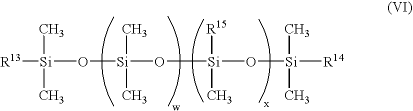
   in which R¹³ and R¹⁴ may represent —OH, R¹⁶ OH or R¹⁷ COOH, independently of each other; R¹⁵ represents —CH₃ or —C₆H₅; R¹⁶ and R¹⁷ represent —(CH₂)y—; y ranging from 1 to 15; w ranging from 1 to 200 and x ranging from 0 to 100.

Among these polysiloxanes modified on at least one end, those bearing at least radical (R¹⁶ and/or R¹⁷) bearing a carboxylic acid group on at least one terminal silicon atom are more preferred.

The fluoroalkyl organosilane compounds (3) may be obtained from fluoroalkyl silanes represented by formula (VII):
CF₃(CF₂)_{z}CH₂CH₂(R¹⁸)ₐSiX₄₋ₐ (VII)
in which:
R represents CH₃—, C₂H₅—, CH₃O— or C₂H₅O —,
X represents CH₃O—or C₂H₅O—,
Z ranges from 0 to 15 and a ranges from 0 to 3.

The fluoroalkyl silanes may be chosen from a non-limiting list especially comprising trifluoropropyltrimethoxysilane, tridecafluorooctyltrimethoxysilane, heptadecafluorodecyltrimethoxysilane, heptadecafluorodecylmethyldimethoxysilane, trifluoropropyltriethoxysilane, tridecafluorooctyltriethoxysilane, heptadecafluorodecyl-triethoxysilane, heptadecafluorodecylmethyldiethoxysilane and the like, in particular trifluoropropyltrimethoxysilane, tridecafluorooctyltrimethoxysilane and heptadecafluoro-decyltrimethoxysilane, and even better still trifluoropropyltrimethoxysilane and tridecafluorooctyltrimethoxysilane.

The silane-based couplers may be chosen from a non-limiting list especially comprising vinyltrimethoxysilane, vinyltriethoxysilane, γ-aminopropyltriethoxysilane, γ-glycidoxypropyltrimethoxysilane, γ-mercaptopropyltrimethoxysilane, γ-methacryloxy-propyltrimethoxysilane, N-β-(aminoethyl)-γ-aminopropyltrimethoxysilane, γ-glycidoxy-propylmethyldimethoxysilane and γ-chloropropyltrimethoxysilane, and the like.

According to a preferred embodiment, the silane coupler includes an aminoresidue in its alkyl chain. Such a residue is particularly useful for binding with the reactive function on the organic colorant like for example the acid function of carmine.

The titanate-based couplers may be chosen from the list comprising isopropylstearoyl titanate, isopropyltris(dioctyl pyrophosphate) titanate, isopropyltris(N-aminoethylaninoethyl) titanate, tetraoctylbis(ditridecyl phosphate)titanate, tetrakis(2,2-diaryloxymethyl-1-butyl)bis(ditridecyl)phosphate titanate, bis(dioctyl pyrophosphate)oxyacetate titanate and bis(dioctyl pyrophosphate)ethylene titanate, and the like.

The aluminate-based couplers may be chosen from acetoalkoxyaluminium diisopropoxide, aluminium diisopropoxymonoethylacetoacetate, aluminium trisethylacetoacetate and aluminium trisacetylacetonate, and the like.

The zirconate-based couplers may be chosen from a list especially comprising zirconium tetrakisacetylacetonate, zirconium dibutoxybisacetylacetonate, zirconium tetrakisethylacetoacetate, zirconium tributoxymonoethylacetoacetate and zirconium tributoxyacetylacetonate, and the like.

The binder may have a molecular mass that may range between 300 and 100 000.

In order to obtain a coat that covers the core uniformly, the binder is preferably in a form that is liquid or soluble in water or in various solvents.

Of these binders, it is preferable to use silicone polymers and derivatives thereof, especially methylhydrogen polysiloxane (KF-99, KF-9901 by Shin-Etsu Silicone) is preferred. Silane coupling reagents and derivatives thereof are also effective binders, especially octyltriethoxysilane and stearyltriethoxysilane are preferred.

Of these binders, it is also preferable to use fluorine-containing binders such as those described in document JP-A-2004-315378, especially heptadecafluorodecyltriethoxysilane [C₈F₁₇C₂H₄)Si(OC₂H₅)₃] is preferred.

Of these binders, it is also preferable to use mixing at least two of different binders. For example, a mixture of γ-aminopropyltriethoxysilane and heptadecafluorodecyltriethoxysilane provides strong repellency against both of water and sebum.

In an exemplary embodiment, the binder is a silicone polymer such as hydroxylmethylpolysiloxane (methicone).

Binding with aid of such a polymer is effective if a colorant such as carmine is mixed and connected with methicone before binding to the surface of the core. In contrast, if methicone is added after finish to bind carmine on the core, methicone makes self-conjugations and insufficiently covers over the surface of carmine-core composite.

Advantageously, mixture of binder and colorant covering the surface of the core is cross-linked. This cross-linkage may be obtained by heating for example by drying the colorant-binder-core composite or by any other convenient method for getting it. After drying the carmine-methicone core, the cross linkage becomes strong bond to store the carmine on surface of the core.

The carmine-methicone-core gives water repellent property which is essential on long wearing. The silicone binder of methicone or the like also gives sebum resistant property because the siloxane polymer has repellency against sebum oil.

The relative quantity of binder may not exceed 10% or 5% of the total weight of the composite pigment.

Surface Treatment Agent

The at least one pigment comprised in the composition of the present invention may be also surface-treated. The surface treatment agent can be added in any process such as premixing of a red organic colorant and a binder or binding process of red organic colorant-binder to the core.

Surface treatment can include processes which have been conventionally performed on cosmetic additives and the like.

For example, the treatment may be chosen from treatments by inorganic metal oxides such as aluminium oxide, magnesium oxide and calcium oxide, and inorganic metal hydroxides such as aluminium hydroxide, magnesium hydroxide and calcium hydroxide (see JP-A-H9-20609 and JP-A-H9-30917); treatments by silic acid, silicon dioxide or inorganic metal salts of silicic acid (see JP-B-H5-74564); treatments by spherical particles such as silica gels, titanium oxide, zinc oxide, magnesium oxide, iron oxide and mica (see JP-B-H7-103324, JP-A-H9-132514 and JP 278461); and silicone polymers, or amino acids such as Nε-lauroyl-L-lysine (see G. Uzunian et al., Cosmetics and Toiletries, Vol. 108, No. 2, pp. 93-98, 1993).

Of these surface treatments, it is preferable to use silicone polymer, especially methylhydrogen polysiloxane (KF-99, KF-9901 by Shin-etsu Silicone) is preferred. This silicone treatment preferably has content of 0.3%-10% of the composition. In contrast, a surface treatment which content is higher than 10% may reduce the smoothness of powder to spread on skin.

Physiologically Acceptable Medium

The composition of the present invention may also contain “physiologically acceptable medium”. The term “physiologically acceptable medium” is used to mean a medium that is not toxic and that is suitable for application to the skin, for example to the face and/or lips, or a keratinous surface, for example the fingernails of human beings. The physiologically acceptable medium is generally adapted to the nature of the medium on which the composition is to be applied, and also to the form in which the composition is packaged.

The cosmetic composition of the present invention may contain other ingredients such as those who are normally used in makeup cosmetic products. Examples include powders, oily ingredients, aqueous ingredients, surfactants, antioxidants, fragrances, preservatives, thickeners, cosmetic active ingredients and polymers and mixtures thereof.

Additional Coloring Substance

A composition according to the invention may include pigments, pearlescent agents and fillers normally used in cosmetics. Pigments include inorganic or organic white or colored pigments.

Examples of inorganic pigments include titanium dioxide, zirconium oxide or cerium oxide which may or may not be surface treated, as well as iron oxide or chrome oxide, manganese violet, ultramarine blue, chrome hydrate or ferric blue. Examples of organic pigments are chosen black, D&C type pigments, and aluminium-, calcium-, strontium-, barium- or cochineal carmine-based lakes.

Pearlescent agents refer to interference substances capable of being selected from among white pearlescent agents such as mica coated with titanium oxide or bismuth oxychloride, and colored pearlescent agents such as titanium oxide-coated mica containing iron oxide, particularly titanium oxide-coated mica containing ferric blue or chrome oxide, titanium oxide-coated mica containing organic pigments, and bismuth oxychloride-based pearlescent pigments.

Oil-soluble colorants includes Sudan red, DC red 17, DC green 6, β-carotene, soybean oil, Sudan brown, DC yellow 11, DC violet 2, DC orange 5 and quinoline yellow. Water-soluble colorants include beet root juice and methylene blue. The overall tone of the composition can be adjusted by blending in the above-described pigments and/or colorants.

Cosmetic active ingredients may include humectants, vitamins, essential fatty acids, sphingolipids or sunscreen agents.

Fatty Phase

The composition of the present invention may contain a fatty phase chosen from among waxes, oils, gums and/or paste-type fatty substances of plant, animal, mineral, synthetic or silicon origin and mixtures thereof, depending on the type of use contemplated.

Examples of waxes which are solid at room temperature include hydrocarbon-containing waxes such as beeswax, camauba wax, candelilla wax, tallow, cork fiber wax or sugarcane wax, paraffin or lignite wax, microcrystalline wax, lanolin wax; montan wax, ozokerite, polyethylene wax, waxes obtained by Fischer-Tropsch synthesis, hydrogenated oils, glycerides which are solid at 25° C. and fatty acid esters. Additionally, silicone waxes can also be used, examples of which include alkoxy and/or esters of polymethylsiloxane. Waxes can be offered in the form of a stable dispersion of wax particles in colloidal form, and can be produced by publicly known methods, such as the method described by L. M. Prince on pages 21-32 of Microemulsions; Theory and Practice, (1997) available from the Academic Press.

Waxes which are liquid at room temperature include jojoba oil and the like.

For example, the fatty phase of the composition may comprise, for example, at least one oil, in particular an oil selected from:
hydrocarbon oils of vegetable origin, such as liquid triglycerides or fatty acids having 4 to 10 carbon atoms and the liquid fraction of butter;
synthetic esters and ethers, in particular of fatty acids, such as oils having formulae R₁COR₂ and R₁OR₂ in which R₁ represents the remainder of a fatty acid having 8 to 29 carbon atoms, R₂ represents an optionally branching hydrocarbon chain containing 3 to 30 carbon atoms, such as for example Purcellin oil, isononyl isononanoate, isopropyl myristate, ethyl-2-hexyl palmitate, octyl-2-dodecyl stearate, isostearate; hydroxyl esters such as isostearyl lactate, octylhydroxystearate, octyldodecyl hydroxystearate, diisostearyl-malate, triisocetyl citrate, heptanoates, octanoates, decanoates of fatty acids; polyol esters such as propylene glycol dioctanoate, neopentylglycol diheptanoate, and diethyeneglycol diisononaoate; and esters of pentaerythritol such as pentaerythrityl tetraisostearate;
linear or branching hydrocarbons of mineral or synthetic origin, such as optionally volatile paraffin oils and derivatives thereof, Vaseline, polydecenes, hydrogenarated polyisobutene, such as parleam oil;
alcohols having 8 to 26 carbon atoms, such as cetyl alcohol, stearyl alcohol, and mixtures thereof (cetylstearyl alcohol), octyldodecanol, 2-butyloctanol, 2-hexyldecanol, 2-undecylpentadecanol, oleic alcohol, or linoleic alcohol;
fluorine-containing silicones such as those described in document JP-A-2 295 912;
silicone oils such as optionally volatile polydimethylsiloxanes (PDMS) having a linear or cyclical silicon chain that are liquid or semiliquid at ambient temperature, in particular cyclopolydimethylsiloxanes (cyclomethicones) such as cyclohexasiloxane; polydimethylsiloxanes including alkyl, alkoxy, or phenyl groups having 2 to 24 carbon atoms; pheny silicones such as phenyltrimethicones, phenyldimethicones, phenyltrimethylsiloxydiphenyl-siloxanes, diphenyldimethicones, diphenylmethyldiphenyltrisiloxanes, 2-phenylethyltrimethyl-siloxysilicates, and polymethylphenylsiloxanes; and
mixtures thereof.

The fatty phase may comprise a film-generating polymer selected from the group comprising:
hydrosoluble film-generating polymers;
aqueous dispersions of hydrodispersible film-generating polymer particles, also known as “latex”, in which case, the composition must include an aqueous phase;
liposoluble film-generating polymers;
lipodispersible film-generating polymers in the form of a non-aqueous of polymer particles, where necessary stabilized on their surface by at least one stabilizing agent in one or more silicone and/or hydrocarbon oils; and
mixtures thereof.
The fatty phase may comprise a wetting agent selected, for example from:
non-anionic wetting agents: fatty acids, fatty alcohols, polyethoxyl or polyglycerol fatty alcohols such as polyethoxylstearyl or cetylstearyl alcohols, esters or fatty acid and sacchorose, alkyl glucose esters, in particular polyoxyethylenated fatty esters of C1-C6 alkyl glucose and mixtures thereof;
anionic wetting agents: C16-C30 fatty acids neutralized by amines, ammonia, or alkali salts, and mixtures thereof.

Emulsion

The composition of the invention may be an emulsion. The proportion of the fatty phase in the composition may lie, for example, in the range 5% to 80% by weight, and in particular 5% to 50% by weight, relative to the total weight of the composition.

By way of example, the composition may comprise an aqueous medium constituting an aqueous phase forming the continuous phase of the composition.

The aqueous phase may be constituted essentially by water. Nevertheless, it may also comprise a mixture of water and organic solvent that is miscible in water, in particular having miscibility in water greater than 50% by weight, such as lower monoalcohols having 1 to 5 carbon atoms such as, for example, ethanol, isopropanol, glycols having 2 to 8 carbon atoms such as propylene glycol, C₃-C₄ ketones, and C₂-C₄ aldehydes.

This aqueous phase comprising in particular water and possibly an organic solvent that is miscible in water, may be present in the composition at a content lying, for example, in the range 1% to 95% by weight, and in particular in the range 3% to 80%, and more particularly in the range 5% to 60% by weight, relative to the total weight of the composition.

As emulsions and co-emulsifiers, mention can be made for example of O/W emulsifiers such as fatty acid esters and polyethylene glycol, in particular PEG-100 stearate, and fatty acid and glycerine esters such as glyceryl stearate, and also W/O emulsifiers such as those available under the trade name ABIL WE09, ABIL EM90, and ABIL EM97 from the supplier Degussa Goldschmidt, or the mixture of ethylene glycol acetyl stearate and glyceryl tristearate sold by the supplier Guardian under the trade name UNITWIX.

As hydrophilic gelling agents, mention can be made in particular of carboxyvinyl polymers (carbomer), acrylic copolymers such as copolymers of acrylates and alkylacrylates, polyacrylamides, polysaccharides, natural gums, clays and mixtures thereof, and as lypophibic gelling agents, mention can be made of modified clays such as bentones, metallic salts of fatty acids, hydrophobic silica, polyethylenes, and mixtures thereof.

Active Agents

The composition of the present invention may comprise active agents chosen, for example, from all the active agents known for their activity on skin aging, such as keratolytic or peering agents, e.g. α-hydroxy acids, β-hydroxy acids, α-ceto acids, β-ceto acids, retinoids and esters thereof, retinol, retinoic acids, and derivatives thereof. Mention can also be made of mixture from veinotonic plants such as extracts of knee-holly and/or horse chestnut; xanthic bases such as cafferine; vitamins, such as for example vitamin A, B1, B2, B3, B5, B6, B12, E, and/or C, and derivatives of such vitamins, and in particular esters thereof; agents against free radicals; sun filters; moisturizers such as polyols; ceramides; DHEA and derivatives thereof; the Q10 coenzyme; bleaching and depigmenting agents such as kojic acid, extracts of scutellaria, mulberry, licorice, and/or chamomile; derivatives of para-aminophenols, of arbutin, and derivatives and mixture thereof.

For use in cosmetically treating fatty or medium skins, the composition of the invention may contain at least one active agent selected, for example, from: vitamins C and B14; zinc salts, in particular zinc oxide and zinc gluconate; salicylic acid and derivatives thereof such as n-octanoyl-5-salicylic acid; triclosan; capryloyglycine ; a clove extract; octopirox; hexamidine; and azelaic acid and derivatives thereof.

It is also possible to include UVA and/or UVB filters in the composition, selected from organic filters and mineral filters, possibly coated in order to make them hydrophobic.

Galenical Form

The composition of the invention may be in the form of an optionally gelled oily solution, an emulsion obtained by dispersing a fatty phase in an aqueous phase (O/W), or vice versa (W/O), a triple emulsion (W/O/W or O/W/O), a vesicular dispersion of ionic type (liposomes or oleosomes) and/or of non-ionic type (niosomes), and/or a dispersion of nanocapsules or nanospheres.

The composition of the invention may be more or less fluid and may have the appearance of a cream, an ointment, a milk, a lotion, a serum, a paste, a form, or a gel, for example. It may also be in solid form, in particular in the form of a stick or a cup or in some other dosage form, in particular in powder form.

The composition may be in a variety of forms, depending on its intended purpose. The composition may thus be in any galenical form which is normally used for topical application, for instance, in the anhydrous form, in the form of an oily or aqueous solution, an oily or aqueous gel, a wax-in-water or a water-in-wax emulsion, a multiple emulsion or a dispersion of oil in water due to the vesicles located on the oil/water interface.

The composition may be in the form of a cast product, such as a stick in the case of a lipstick or a lip care product. The composition may also be in a variety of other forms, for example a liquid of greater or lesser viscosity, a gel or a paste. The composition may also be a bar to be moistened for use, to allow it to disintegrate.

### EXAMPLES

Conditions of Measures and Protocols

Method-1; Carmine and filler pigment without binder was prepared as follows. A 100 g of core filler such as mica and 1 g of carmine was added into 1 1 of distilled water and pH of the mixture solution was once increased to 8.5 to activate the carboxyl residue of carmine by adding 0.5 N NaOH solution. The mixture was strongly stirred for 1 h with a six bladed impeller. After that, the pH of the mixture solution was neutralised by adding aqueous HCl (0.5N). The neutralised solution was recovered and filtrated via a No 5C filtration paper. The wet pellet of carmine-filler composite was recovered by filtration, and was diluted again in a 500 ml of hot water and stirred for ten minutes for washing. The washed carmine-filler was recovered by filtration. The washing of carmine-filler was repeated once more. After that, the wet pellet of carmine-filler composite recovered by filtration was heated at 130° C. The dried bulk was milled and sieved before testing the characteristics as cosmetic fillers.

Method-2; Carmine and filler pigment, coated with binder was prepared as follows. A carmine-filler prepared by method 1 was dispersed in 1 1 of distilled water and 6 g of mixture solution composed of 2 g of methicone and 4 g of isopropanol was added into the carmine-filler solution. The mixture was strongly stirred for 1 h with a six bladed impeller at room temperature. After the agitation, the wet pellet of carmine-filler adsorbed with methicone was recovered by filtration and heated at 130° C. for 24 h to fix methicone on the surface of carmine-filler by dehydrogen and dehydration reactions. The dried bulk was milled and sieved before testing the characteristics as cosmetic fillers.

Method-3; Filler pigment coated with carmine-binder mixture was prepared as follows. A mixture of carmine and methicone binder was prepared by adding 1 g of carmine in a solution composed of 2 g methicone and 6 g of isopropanol and stirred for 1 h at room temperature. After that, the mixture of carmine and methicone solution was added into a solution of 100 g of core filler dispersed in 1 1 of distilled water. The mixture was strongly stirred for 1 h with a six bladed impeller. After the agitation, the wet pellet of carmine-methicone-filler was recovered by filtration and heated at 130° C. for 24 h to fix methicone and carmine on the surface of filler by dehydrogen and dehydration reactions. The dried bulk was milled and sieved before testing the characteristics as cosmetic fillers.

Stability of the carmine fixed at the pigment was tested by washing the dried powder of carmine-filler complex with hot water. The red color of the pigment was compared between before and after the washing.

A transparent double-sided adhesive tape having an area of 3×3 cm² adhered to white paper, and 30 mg of the pigment were applied to the adhesive surface using a puff for foundations.

The reflectance of this sample was measured using a color meter (Minolta). The color meter was set to computer and the average value for the three locations was taken as the measurement value of the sample.

The resulting measurements were inputted into the color analysis software (Core Science) to form spectral reflectance curves.

Contact angles of water and oleylolerate against the pigment were measured by putting drops of water and oleyloleate on the pigment spread on the adhesive tape.

Aspect ratio, diameter/thickness, of core filler was determined by measuring the diameter and thickness of each particle taken by scanning electron microphotograph.

Adhesion test of pigment on plastic film was carried as follows. A 10 g of pigment and a piece of plastic film having an area of 3×3 cm² was shaken for three minutes in a plastic bug. Then the plastic film was recovered and the weight of the pigments adhered on the both surface of film was measured. After that, the color value of a* of the pigment adhered on the plastic film was measured by use of CR-200 (Minolta).

Carmine Composite Core

Various cores are used for preparation of carmine composite powders.

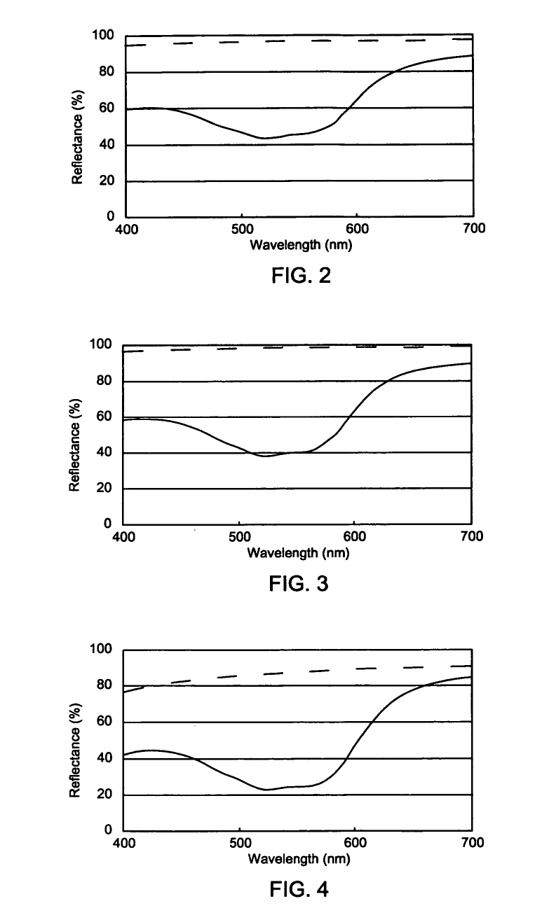

FIG. 2 shows the spectral reflectance curves of Boron nitrite (dash line) and Carmine-Boron nitrite (full line). The curve of Carmine-Boron nitrite has the similar pattern with the model curve of the “personal beautiful skin”, that is, a broad peak in a wavelength region (region I) of 450-550 nm, a trough in a lower wavelength region (region II) of 500-620 nm, and gradually increasing in the wavelength region (region III) of 600-700 nm. In contrast, the original powder of Boron nitrite without carmine shows no peak and no trough in the regions.

FIG. 3 shows the similar results of spectral reflectance curves of BaSO₄ (dash line) and its corresponding carmine composite.

Furthermore, FIG. 4 shows the spectral reflectance curves of Coverleaf AR80® (dash line), a composite of TiO₂, Al₂O₃, SiO₂ and talc, and its carmine composite (full line).

As shown in FIG. 4, even in the complex composite powder, the carmine treatment gives the typical pattern of the “personal beautiful skin”. This typical pattern is also obtained in any core material such as mica, synthetic mica, sericite, silica and talc, when the core is modified with carmine.

While the similar pattern is obtained in each carmine composite powder, the brightness is varied depending on the core.

As shown in FIGS. 2, 3 and 4, the reflectance ratio by the carmine composite at the trough region II is higher by use of the Boron nitrite than by use of carmine-Coverleaf. These results show that the brightness varies depending on the reflectance of the core material, while the typical pattern of the “personal beautiful skin” is obtained by modifying various cores with carmine. Thus, the carmine-core gives moderate brightness on skin by eliminating strong glitter and reflects natural appearance of the makeup finish beside the “personal beautiful skin”.

Carmine Composite Core With a Binder

Table 1 below summarizes three examples. In each example, comparative examples are also submitted.

Contact angles of water and sebum-like oil on the surface of each red organic colorant-core composite are summarized in Table 2. Oleylolerate is used as the sebum-like oil. The contact angle of water on the Carmine-core composite is always higher than 125° with a binder. In contrast, the contact angle of water is very low in comparative examples without binders. These results show that the binder gives hydrophobic property to the carmine-core composite.

Contact angle of oil also shows that it is always lower than 20° if there is no binder. Silicone binders are also certain effective on enhancing the oil repellence as in examples except for octlysilane. The oil repellent property becomes stronger if comprising binder having perfluoro residues such as heptadecafluorodecyltriethoxysilane (JP-A-2004-315378). Thus the sebum repellency is also enhanced by use of siloxane polymers, a binder having perfluoro residues, and the like for the carmine-core composite.

These repellence against water and oil shows that the binder is beneficial for improving long wearing, since the sebum and sweat cause the decay of makeup.

The powders treated with a binder after composing carmine-core give high water repellence and oil repellence as well as the results of examples 1, 2 and 3. However, the red color of the composite core is considerably removed in cases of 1b, 2b, and 3b, while the color is stably preserved even after the washing in hot water in examples 1, 2 and 3.

These results show that the premixing of carmine with binders provides stable conjugation between carmine and the core and, therefore, effective on long wearing without removal of the carmine coated on the core.

TABLE 1
Composition of carmine-core composite powder

| | Core | Binder | Colorant |
|---|---|---|---|
| Example | | | |
| 1 | TiO₂-mica | Methicone-1 | Carmine |
| 2 | Silica | Methicone-2 | Carmine |
| 3 | Synthetic mica | Silane-3 | Carmine |
| Comparison | | | |
| 1a | TiO₂-mica | | Carmine |
| 2a | Silica | | Carmine |
| 3a | Synthetic mica | | Carmine |
| 1b | TiO₂-mica | Methicone-1* | Carmine |
| 2b | Silica | Methicone-2* | Carmine |
| 3b | Synthetic mica | Silane-3* | Carmine |

Methicone-1: KF-9901 Methylhydrogenopolysiloxane from Shin-etsu coded E201232
Methicone-2: KF-9901 + octyltriethoxysilane
Silane-3: octyltriethoxysilane
*the binder was added after composing carmine-core composite without binder, i.e. according to the previous method 2.

TABLE 2

| | Contact | | | | |
|---|---|---|---|---|---|
| | angle | | Red (a*-value) level | | |
| | Water | oil | before wash(B) | after wash(A) | (A)/(B) % |
| Example | | | | | |
| 1 | 138 | 48 | 9.4 | 9.2 | 98 |
| 2 | 132 | 30 | 12.3 | 12.1 | 98 |
| 3 | 135 | 10 | 13.7 | 12.4 | 91 |
| Comparison | | | | | |
| 1a | 105 | 18 | 10.1 | 6.5 | 64 |
| 2a | 8 | 15 | 13.5 | 6.2 | 46 |
| 3a | 30 | 19 | 14.2 | 4.5 | 32 |
| 1b | 135 | 43 | 9.6 | 7.0 | 73 |
| 2b | 125 | 28 | 12.7 | 9.5 | 78 |
| 3b | 130 | 10 | 13.1 | 8.4 | 64 |

### Examples of Makeup Compositions

The formulation elements given below are presented purely by way of illustration, and percentages are expressed throughout by weight relative to the total weight of the composition.

The compositions are prepared using conventional cosmetic preparation methods.

### Example 4: Cream Foundation O/W

The cosmetic composition of Example 4 comprises sericite with carmine and a binder in an O/W type cream foundation. Methicone is used as the binder. The pigment is in the form of flakes having a sericite-carmine-binder and with a weight ratio of 96.5%/0.5%/3%, with flakes having a mean size of 9 μm.

Foundations are obtained with the compositions shown in the Table 3 (the numerical values in Table 3 are expressed in % by weight). Comparative Example 4a is prepared by a non-treated sericite instead of the sericite-carmine-binder composite. The cream foundation of Example 4 gives natural and translucent finish compared to the Comparative Example 4a. The result shows that the sericite is also an effective core for composing carmine composite to restore the “natural and personal beautiful skin” even in the cream foundation.

TABLE 3
Composition of cream foundation O/W

| | | | Comp. |
|---|---|---|---|
| | | Ex. 4 | Ex. 4a |
| Phase | Raw Materials | (%) | (%) |
| A | Purified water | 70.2 | 70.2 |
| | Glycerin | 2 | 2 |
| | Preservative | 0.8 | 0.8 |
| B | Sepigel 305 (Seppic) | 2 | 2 |
| C | Cyclopentasiloxane | 12 | 12 |
| | Dimethiconol | 0.5 | 0.5 |
| D | C9-15 Fluoroalcohol Phosphate Treated Yellow | 0.77 | 0.77 |
| | Iron Oxide | | |
| | C9-15 Fluoroalcohol Phosphate Treated Red | 0.11 | 0.11 |
| | Iron Oxide | | |
| | C9-15 Fluoroalcohol Phosphate Treated Black | 0.05 | 0.05 |
| | Iron Oxide | | |
| | C9-15 Fluoroalcohol Phosphate Treated | 5.07 | 5.07 |
| | Titanium Dioxide | | |
| | C9-15 Fluoroalcohol Phosphate Treated | 3 | 3 |
| | Titanium Dioxide | | |
| E | Tocopherol Acetate | 0.5 | 0.5 |
| F | Sericite-carmine-binder | 3 | |
| | Non-treated Sericite | | 3 |
| | Total | 100 | 100 |

### Example 5: Liquid foundation W/O

Foundations are prepared with the compositions shown in the Table 4. Comparative Example 5a is prepared by comprising non-treated sericite instead of the sericite-carmine-binder composite. The liquid foundation of Example 5 gives natural and translucent finish compared to the comparative Example 5A.

The result shows that the carmine-core composite is highly effective for providing “natural and personal beautiful skin” when it is applied in liquid foundation.

TABLE 4
Composition of liquid foundation W/O

| | | | Comp. |
|---|---|---|---|
| | | Ex. 5 | Ex. 5a |
| Phase | Raw Materials | (%) | (%) |
| A | Cetyl PEG/PPG-10/1 Dimethicone | 0.8 | 0.8 |
| | Polyglyceryl-4 Isostearate | 0.6 | 0.6 |
| | Hexyl Laurate | 0.6 | 0.6 |
| B | PEG-10 Dimethicone | 5 | 5 |
| | Cyclohexasiloxane | 40.58 | 40.58 |
| | Preservative | 0.15 | 0.15 |
| C | Disteardimonium Hectorite | 1.6 | 1.6 |
| D | C9-15 Fluoroalcohol Phosphate Treated Yellow | 0.96 | 0.96 |
| | Iron Oxide | | |
| | C9-15 Fluoroalcohol Phosphate Treated Red | 0.15 | 0.15 |
| | Iron Oxide | | |
| | C9-15 Fluoroalcohol Phosphate Treated Black | 0.03 | 0.03 |
| | Iron Oxide | | |
| | C9-15 Fluoroalcohol Phosphate Treated | 7.33 | 7.33 |
| | TiO₂-mica | | |
| E | PMMA | 4 | 4 |
| F | Purified Water | 23.55 | 23.55 |
| | Glycerin | 10 | 10 |
| | Sodium Chloride | 0.7 | 0.7 |
| | Preservative | 0.95 | 0.95 |
| G | Sericite-carmine-binder | 3 | |
| | Non-Treated Sericite | | 3 |
| | Preservative | 1 | 1 |
| | Total | 100 | 100 |

### Example 6: Powder Foundation

The cosmetic composition of Example 6 comprises sericite with carmine and a binder in a powder cake foundation. The pigment is in the form of flakes having a sericite-carmine-binder, and with a weight ratio of 95.5%/0.5%/4%, with flakes having a mean size of 9 μm. The carmine and methicone binder are premixed before binding to sericite in Example 6. Foundations are prepared with the compositions shown in the Table 5. Comparative Example 6A is also prepared by comprising sericite-carmine and binder in two steps, sericite-carmine//binder. The sericite-carmine//binder was prepared by modifying in two steps with methicone after binding carmine to sericite, instead of the one step modified sericite-carmine-binder composite.

Foundation of Example 6 comprising sericite-carmine-binder gives a natural and healthy finish. Translucency and brightness in appearance are also obtained by applying the foundation of Example 6. The natural and healthy looking is maintained for a long time without dullness in color and translucency on face.

Comparative Example 6A gives similar makeup results in attributes of natural, healthy, translucency and brightness as those obtained by Example 6. However, comparative Example 6A is insufficient in anti-dullness and long lasting compared to the Example 6. These results show that the sericite-carmine-binder composite obtained in the present invention is highly effective on long lasting while sericite-carmine//binder composite by the previous method is weak on long lasting.

TABLE 5
Composition of powder cake foundation

| | | | Comp. |
|---|---|---|---|
| | | Ex. 6 | Ex. 6a |
| Phase | Raw Materials | (%) | (%) |
| A | TiO₂ | 5 | 5 |
| | Yellow Iron Oxide | 2.4 | 2.4 |
| | Red Iron Oxide | 0.45 | 0.45 |
| | Black Iron Oxide | 0.15 | 0.15 |
| | Sericite-carmine-binder | 30 | |
| | Sericite-carmine//binder | | 30 |
| | TiO₂-mica | 3 | 3 |
| | Talc | 33.5 | 33.5 |
| | Mica | 10 | 10 |
| | Silica | 5 | 5 |
| | Zinc Myristate | 1.5 | 1.5 |
| B | Squalane | 8 | 8 |
| | Preservative | 1 | 1 |
| | Total | 100 | 100 |

Needless to say, the invention is not limited to the working examples that have been given.

It is possible to use composite pigments according to the present invention to prepare cosmetic compositions having formulations other than those given above.

The ranges given should be understood as being inclusive of the limit, unless the opposite is specified.

Aspect Ratio for Adhesion

Table 6 summarized the adhesion of various pigment to plastic film. The red color reflection is also summarized. Elongated core with an aspect ratio higher than 40 was effective on adhesion of pigment, while thick pigment with less than 20 showed the less adhesiveness. Beside this, the higher aspect ratio than 20 was effective on reflection of red color against the specific area. These results show that the elongated core with higher aspect ratio is essential for adhesion of pigment and reflecting its color on skin.

TABLE 6
Adhesion and reflection of red of pigment

| Ex- | | | | | | |
|---|---|---|---|---|---|---|
| am- | | Size | Thick | Aspect | Adhesion | Red |
| ple | Core | (μm) | (μm) | ratio | (mg/cm²) | (a*/cm²) |
| 7 | Talc A | 9.5 | 0.56 | 17 | 0.25 | 0.78 |
| 8 | Synthetic mica B | 12.0 | 0.55 | 22 | 0.33 | 1.12 |
| 9 | Synthetic mica C | 6.7 | 0.16 | 42 | 0.83 | 1.28 |
| 10 | Synthetic mica D | 11.6 | 0.20 | 58 | 0.89 | 1.26 |
| 11 | Mica E | 7.9 | 0.14 | 56 | 0.51 | 1.26 |
| 12 | Mica F | 21.8 | 0.25 | 87 | 1.78 | 1.29 |

Although the present invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. Cosmetic composition comprising:
at least one pigment comprising:
an elongated core,
a red organic colorant coating at least partially the core, and
at least one binder between the core and the organic colorant.

2. The composition according to claim 1, wherein the core has an aspect ratio not less than 20.

3. The composition according to claim 2, wherein the aspect ratio is not less than 30.

4. The composition according to claim 1, wherein the red organic colorant has a red color a* in the CIE L*a*b* color space ranging from 5 to 45.

5. The composition according to claim 1, wherein the at least one binder derives from one compound which molecule(s) may include at least two active sites to bind respectively with the colorant and the core.

6. The composition according to claim 5, wherein the binder is based on at least a silicone compound.

7. The composition according to claim 1, wherein the binder is cross-linked.

8. A cosmetic composition according to claim 1, wherein the organic colorant is a natural colorant.

9. A composition according to claim 8, wherein the organic colorant is carmine.

10. The composition according to claim 9, wherein the organic colorant is an anthraquinone-type colorant.

11. The composition according to claim 1, wherein the organic colorant is a synthetic organic colorant chosen amoung dyes including azo dyes such as RED No.505 and xanthan dyes such as RED No.230 and RED No.233, RED No. 228, lakes such as RED No.202 and organic pigments such as RED No.228 or mixtures thereof.

12. The composition according to claim 1, wherein the organic colorant is a natural organic colorant chosen among carotenoide colorants such as Carthamin, flavonoid colorants including anthocyanidins and chalcones, quinine colorants including anthraquinones and naphthoquinones, and betacyanidin colorants such as betanin, or mixtures thereof.

13. The composition according to claim 1, wherein the core is an inorganic core.

14. The composition according to claim 12, wherein the inorganic core includes at least one of: metal salts, metal oxides, alumina, glasses, ceramics, graphite, silica, silicates, mica, synthetic mica, talc, sericite, boron nitrite, barium sulphate, clays, synthetic clays, and mixtures thereof.

15. The composition according to claim 1, wherein the core is an organic core.

16. The composition according to claim 1, wherein there is an intermediate layer between the organic colorant and the core.

17. The composition according to claim 16, wherein the intermediate layer includes at least one metal oxide.

18. The composition according to claim 17, wherein the at least one metal oxide is chosen from titanium dioxide, zinc oxide, zirconium oxide, cerium oxide, iron oxide, aluminium oxide, chrome oxide, calcium oxide, silica, and magnesium oxide.

19. The composition according to claim 1, wherein the core has a mean size of about 1-40 μm.

20. The composition according to claim 1, wherein the core has a mean size of about 5-20 μm.

21. The composition according to claim 1, wherein the binder is based on at least a compound selected from the group: silicone polymers and derivatives thereof, methylhydrogen polysiloxane, silane couplers and derivatives thereof, octyltriethoxysilane and stearyltriethoxysilane, fluorine containing silicone, mixture of at least two of different couplers, for example a mixture of γ-aminopropyltriethoxysilane and heptadecafluorodecyltriethoxysilane.

22. The composition according to claim 1, comprising a fatty phase chosen among waxes, oils, gums, and/or paste-type fatty substances of plant, animal, mineral, synthetic or silicone origin and mixtures thereof.

23. The composition according to claim 1 comprising a fatty phase, characterized by the fact that it is an emulsion, the proportion of the fatty phase in the composition lying in the range 5% to 80% by weight, relative to the total weight of the pigment of said composition.

24. The composition according to claim 22, wherein the proportion of the fatty phase in the composition lie in the range 5% to 50% by weight.

25. The composition according to claim 1, wherein the content of the red organic colorant is 0.1%-5% by weight, relative to the total weight of the pigment comprised in the composition.

26. The composition according to claim 1, wherein the red organic colorant is carmine and the binder is a silicone binder.

27. A composition according to claim 26, wherein the core is sericite.

28. A composition according to claim 26, wherein the binder has perfluoro residues.

29. A composition according to claim 28, wherein the binder is based on at least heptadecafluorodecyltriethoxysilane.

30. The composition according to claim 26, wherein the binder is based on at least methicone.

31. The composition according to claim 26, wherein the weight ratio of carmine ranges from 0.1% to 5%, the weight ratio of binder ranges from 0.3% to 10% and the weight ratio of sericite ranges from 85% to 99.6%.

32. The composition according to claim 1, wherein the core is sericite, the colorant is carmine and the percentage by weight, relative to the total weight of the composition, of sericite-carmine-binder ranges from 0.5% to 90%.

33. The composition according to claim 1, which spectral reflectance curve of the made-up skin has a trough in a lower wavelength region of 500-620 nm.

34. The composition according to claim 1, wherein the quantity of binder does not exceed 10% of the total weight of the pigment.

35. The composition according to claim 1, wherein the quantity of binder does not exceed 5% of the total weight of the pigment.

36. The composition according to claim 1, comprising at least one surface treatment agent.

37. The composition according to claim 36, wherein the at least one surface treatment agent is a silicone polymer.

38. The composition according to claim 36, wherein the quantity of surface treatment agent ranges from 0.3% to 10% of the total weight of the pigment of said composition.

39. The composition according to claim 1, which is a foundation.

40. A kit comprising:
a first cosmetic composition according to claim 1, and
a second cosmetic composition, the two compositions being separately packaged and configured to be applied one to the other.

41. A cosmetic method comprising application to the skin of a cosmetic composition according to claim 1.

42. A method of making a cosmetic composition, wherein an organic colorant is premixed and connected with a binder in a first time, and in a second time, the core is added to the organic colorant-binder.

43. A method of making a cosmetic composition, wherein the surface of a core is treated by a premixed organic colorant connected to a binder.

44. The method according to claim 42, wherein the organic colorant is carmine, the binder is based on at least a silicone compound and the core is sericite.

45. The method according to claim 44, wherein the weight ratio of carmine ranges from 0.1% to 5%, the weight ratio of binder ranges from 0.3% to 10% and the weight ratio of sericite ranges from 85% to 99.6%.

46. The method according to claim 43, wherein the core is sericite, the colorant is carmine and the percentage by weight, relative to the total weight of the composition of sericite-carmine-binder ranges from 0.5% to 90%.
